(19) United States
(12) Patent Application Publication
(10) Pub. No.: US 2007/0074989 A1
(43) Pub. Date: Apr. 5, 2007

(54) Container for lyophilization and storage of tissue

(75) Inventors: Barbara L. Merboth, Bridgewater, NJ (US); Florence Stoffel, Flemington, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/239,342

(22) Filed: Sep. 30, 2005

(51) Int. Cl. A61B 19/02

(52) U.S. Cl. 206/438; 206/471

(57) ABSTRACT

A sterile container assembly for storing sterile allograft tissue implant material is constructed with sidewalls, end walls and a base member defining an open faced cavity and a flange surrounding and extending outward from the cavity. A step and a spacer is formed in each end wall of the container and a cylindrical implant container sized to fit into the container cavity is positioned adjacent the end wall spacers. The implant container has a housing and a threadable cap with a vent hole formed therein which is covered by an insert member constructed of sintered PTFE mounted inside the cap. A foil cover is sealed to the flange of the container covering and sealing the container cavity and the cylindrical implant container mounted therein.

### CROSS-REFERENCE TO RELATED APPLICATIONS

There are no other applications related to the present application.

### TECHNICAL FIELD OF THE INVENTION

The present invention generally relates to packaging for sterile tissue specimens for use in tissue transplant and more specifically to packages for allograft implant tissue forms in a specifically designed sterile package or container which allows the implant to be effectively lyophilized while preventing cross contamination by lipids or microbial agents released from the tissue during lyophilization or storage.

### BACKGROUND OF THE PRIOR ART

Allograft tissue forms are useful in orthopaedic and neurosurgery. In practice, processed human tissue is delivered to the hospital and eventually to the surgeon in a form useful for surgical implantation in a sterile package. Unfortunately, while large amounts of time have been spent on development and processing of tissue implant forms and materials, little consideration has been spent on the package design and the specific problems involved with packaging bone tissue material. Packaging that is currently used for bone implant forms is cumbersome and it is often difficult to easily remove the implant form from such packaging while wearing gloves during a surgical procedure.

Furthermore, a need has developed for a simple, inexpensive packaging that may be used to safely retain implant allograft bone tissue material in a sterile condition away from the lipid pooling while allowing storage of same and maintaining sterility up until the actual time of implantation during surgery. While tissue processing removes many of the lipids found in bone, some will remain in the tissue, particularly in cancellous tissue. Over time these lipids drain from the tissue and are deposited onto the package. The lipids can be absorbed by or cause discoloration of the packaging material. If the package material forms part of the package seal and that material absorbs the lipids the seal will be destroyed causing failure of the sterile barrier. When the material is only discolored or does not form part of the sterility seal, the result is a visually unattractive package, which while causing no harm to the package or tissue gives the appearance that the product is damaged or spoiled.

There are presently available a number of kinds of packaging for sterile specimens. One form of commonly used packaging is to provide the allograft tissue in a freeze-dried state in a glass jar or bottle with a specifically designed stopper. The aseptically processed tissue is placed into the glass jar, and the stopper is placed on top of the jar. The stopper is designed so that it sits on top of the jar and there is a gap that allows the moisture to escape during the lyophilization process. The jars are then placed into a commercial freeze dryer with the freeze drying process occurring under vacuum. At the end of the cycle while the jars are still under vacuum, a mechanism in the freeze dryer presses the stopper into the jar and creates a seal between the jar and the stopper that maintains the vacuum in the jar. The jars are then removed from the freeze dryer and the closed stopper is secured in place with a metal crimp and plastic lid. This package is effective in allowing the moisture to be removed from the tissue, and in protecting the tissue. However there are several drawbacks to this design. The first drawback is since the stopper is in the up position for lyophilization and since many donors are processed in one freeze drying cycle, there is a potential for cross contamination between containers. Secondly, the jars are subject to breakage during shipping and thirdly, the metal crimp cap is difficult to remove and can tear the gloves of the operating room personnel opening the container, causing possible contamination and the need to destroy the tissue.

Another form of packaging which has been used for holding products such as a pre-sterilized medical devices and allograft implant forms are sealed blister containers. Such containers generally comprise a relatively rigid blister tray with a peripheral flange and a foil composite material or paper backing sheet positioned over the open tray in overlapping engagement with the peripheral flange forming a cover. The cover is manually peeled away from the flange to which it is sealed to allow access to the medical device or implant tissue form contained therein. Other examples of packaging for medical devices or implant forms which have been utilized or are known in the art are shown in the following patents.

U.S. Pat. No. 6,012,580 is directed toward a clamshell type package with two halves pivoted together at a hinge constructed of a transparent plastic constituted to serve as a universal package for implant materials.

U.S. Pat. No. 5,720,391 shows a blister package and insert holder for a heart valve prosthesis. The package is constructed with an outer tray which receives an inner tray. An inner tray lid seals the inner tray and is provided with a pull tab on its exterior surface so that the same can be pulled away from the lip of the inner tray for access to the sterile heart valve prosthesis. The outer tray is provided with a tray lid which is sealed to the lip of an outer flange of the outer tray.

U.S. Pat. No. 5,690,226 shows a sealed air tight molded blister package of PETG having a hollow interior with the opening surrounded by an outwardly extending flange. A multi-layer peelable cover is sealed to the flange surrounding the opening. The cover and the flange are deformed towards the bottom of the container from the plane of the opening sealed by the peelable cover.

U.S. Pat. No. 5,615,770 discloses a sterilizable medical implant package insert placed within a standard sterile implant package holding an implant and allowing for the automatic presentation of the implant from the insert when the sterile package top is opened.

U.S. Pat. No. 5,257,692 is directed toward a three envelope package for preserving tissue specimens or other sterile objects. The sterile tissue sample is sealed within an innermost envelope which is sterile inside and outside. The innermost envelope is sealed within the sterile interior of an intermediate envelope which is both sterile inside and outside. This intermediate envelope is made of foil or another substance impermeable to a storage medium such as liquid nitrogen and is sealed within the sterile interior of an outermost envelope made of foil or another substance impermeable to a storage medium. The outermost envelope provides complete impermeability to liquid nitrogen, eliminating the possibility of nitrogen seepage through the peel-baked seal of the intermediate envelope.

U.S. Pat. No. 5,176,258 shows a package with a peripheral flange around a blister defining an open cavity for receiving a product and a compressible insert for securing the product against movement in the package cavity. At least one projection on the insert extends laterally from the cavity over the peripheral flange and a lid covering the cavity is continuously sealed to the peripheral flange and the projection along a single seal.

U.S. Pat. No. 4,750,619 is directed toward a package for a sterile prosthetic implant element comprising an outer receptacle and an inner receptacle which fits into the cavity of the outer receptacle. Both of the receptacles are provided with outwardly extending flanges surrounding the outer periphery of the cavity of each receptacle and receive a lid which is secured thereto. A tray received within the inner receptacle has hinged leaves so that upon placement of the tray within the receptacle and folding the hinged leaves, the prosthetic implant element is confined within a defined envelope to protect the same against damage.

U.S. Pat. No. 4,697,703 discloses a double sterile package for medical items such as a hip joint prosthesis with an outer open container enclosing an inner package containing the medical item. The inner package has a first insert contained in the open top container, a lid and a second insert carried by the lid. The outer container is sealed with a cover which engages an outer peripheral flange formed around the open top of the outer container.

U.S. Pat. No. 6,830,149 by the present inventor discloses a package for storing sterile allograft tissue implant forms constructed with an outer container defining an open faced cavity and a flange extending outward from said cavity with a stepped recess formed in said flange surrounding the cavity. An inner container which is adapted to be seated in the cavity of the outer container defines an open faced cavity and a flange extending outward from said cavity, the inner container flange being of a dimension to fit into the stepped recess of the outer container. An insert member sized to fit into said inner container cavity defines a shaped depression therein to hold a tissue implant form within a designated space defined by the shaped depression. The inner container is covered by a permeable cover sealed to the flange of the inner container allowing the tissue form to be treated and the outer container is covered by an impermeable cover sealed to the flange of the outer container covering the outer container cavity. This design is currently used for tissue forms that are not in the powder, chip or granular form. It is not effective for powder, chips and granules because the venting required in the retainers allows the small particles to move from under the retainer and fall on the TYVEK® lid. In addition to poor presentation of the product, the lipids in the tissue could then damage the seal.

None of the above noted packages are conducive to bone tissue forms in the powder, chip or granular form. Human bone tissue forms, after processing, retain lipids sometimes called structural fats. Lipids are a group of fatty substances that include fatty oils, waxes, sterols such as cholesterol, triglycerides (the principal forms of fat in body fat) and esters of fatty acids containing groups such as phosphoric acid (phospholipids) and carbohydrates (glycolipids). Over time, the fatty oils contained in the processed bone material are drawn out of the bone and are absorbed by the permeable cover and begin to dissolve the adhesive bond between the cover and the container. This activity causes the cover to separate from the container receptacle destroying the sterility of the package and leaving pools or rivulets of a dark oily material which is unsightly and causes the surgeon to discard or send back the tissue material. This lipid migration is a serious problem for manufacturers of allograft bone material, and presents problems for the hospital and surgeon user. Accordingly, the present container assembly has been developed for lyophilization of allograft bone material in which during lyophilization, venting occurs through a microbial barrier which prevents microbes and lipids from escaping or entering the container. The package does not need to be opened to facilitate later packaging or storage steps, thus further preventing cross-contamination or potential loss of sterility.

### SUMMARY OF THE INVENTION

The present invention is directed toward an allograft tissue package constructed to hold allograft bone tissue material in a plastic bottle mounted in a sterile blister package allowing drainage of the lipids contained in the implant during lyophilization without destroying the seal and sterility of the package. The package for storing sterile allograft tissue implant forms is constructed with an outer container defining an open faced cavity and a flange extending outward from said cavity and surrounding the cavity. A cylindrical implant material container which is adapted to be seated in the cavity of the container is formed with a cylindrical jar housing with a screw on cap having a vent hole and a thin porous cylindrical insert member mounted in the cap.

It is an object of the invention to provide for lyophilization of bone tissue material while the same is contained within the package container while preventing microbes from escaping or entering the container and to resist absorption and discoloration from lipids found in the bone tissue material.

It is another object of the invention to provide a packaging system which holds sterile bone tissue material in a sterile condition which allows a user easy access to the sterile bone tissue material.

It is yet another object to provide a sterile package for bone tissue material which is resistant to cracking or shattering or loss of strength during the freeze drying process and during storage.

It is another object of the invention to provide a medical package in which an implant tissue form can be maintained in a sterile condition when it is presented to the surgeon for implantation.

It is still another object of the invention to provide a package for storing sterile bone tissue material for later use in an implant situation.

It is yet another object of the invention to provide a package for storing bone tissue implant material whereby the bone tissue implants material can be removed from the package in a sterile condition.

It is still another object of the invention to provide a sterile bone tissue package which prevents cross contamination while being lypholized and stored.

It is yet another object of the invention to provide a sterile tissue package which is easy to use during the processing phase and by the end user in the operating room.

In the accompanying drawings, there is shown illustrative embodiments of the invention from which these and other objectives, novel features and advantages will be readily apparent.

### BRIEF DESCRIPTION OF THE DRAWINGS

FIG. 1 is a perspective view of the sterile allograft tissue package;

FIG. 2 is an exploded perspective view of the tissue package of FIG. 1 showing the cover, the tissue container and the blister pack in spaced relationship;

FIG. 3 is top plan view of the blister pack of the tissue package;

FIG. 4 is a side elevation view of the blister pack of FIG. 3;

FIG. 5 is a side elevation view of the tissue container with attached cap;

FIG. 6 is an exploded view of the tissue container of FIG. 5;

FIG. 7 is a top plan view of the container cap with the insert member removed;

FIG. 8 is a top plan view of the insert member;

FIG. 9 is a side elevation view of the insert member; and

FIG. 10 is a top plan view of the cap with insert member inserted therein.

### DETAILED DESCRIPTION OF THE INVENTION

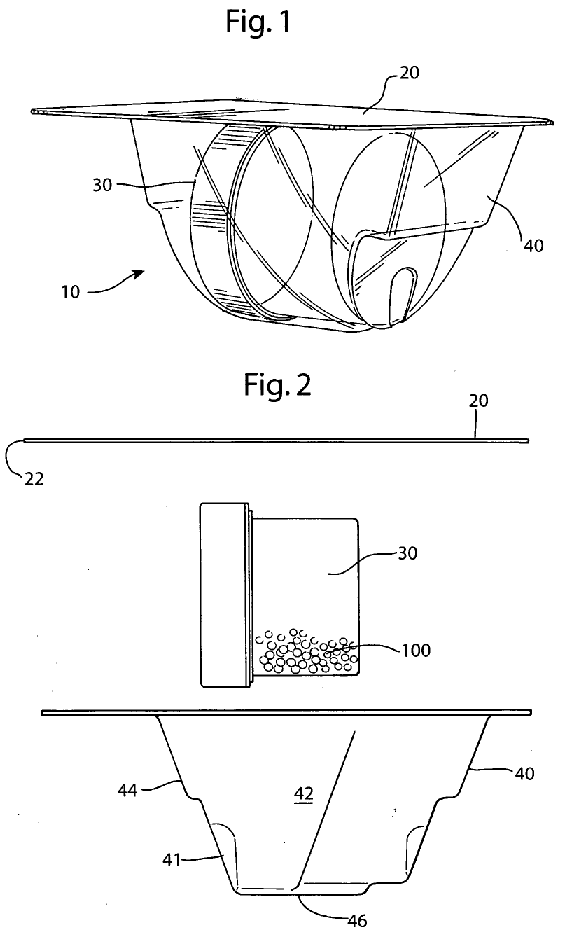

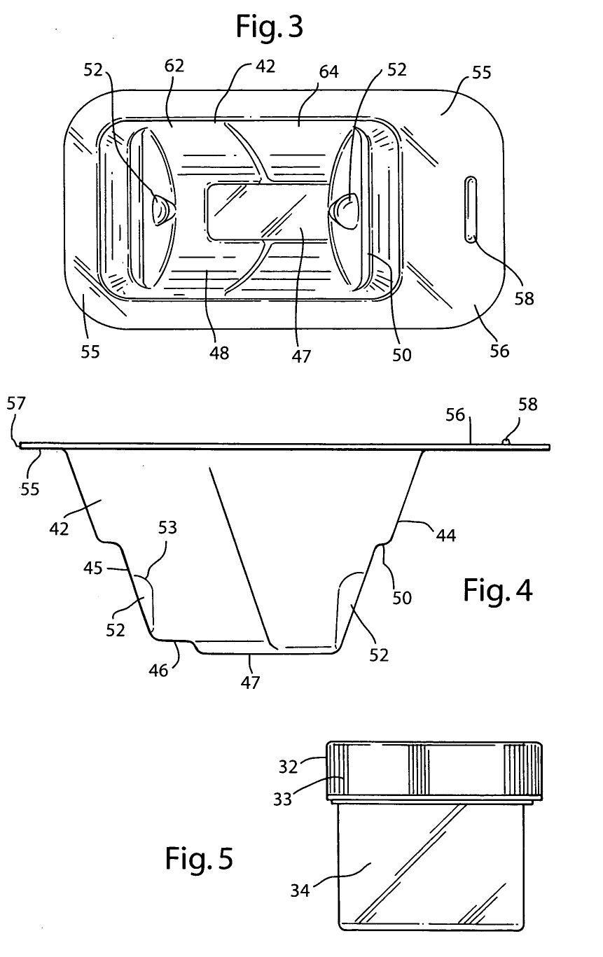

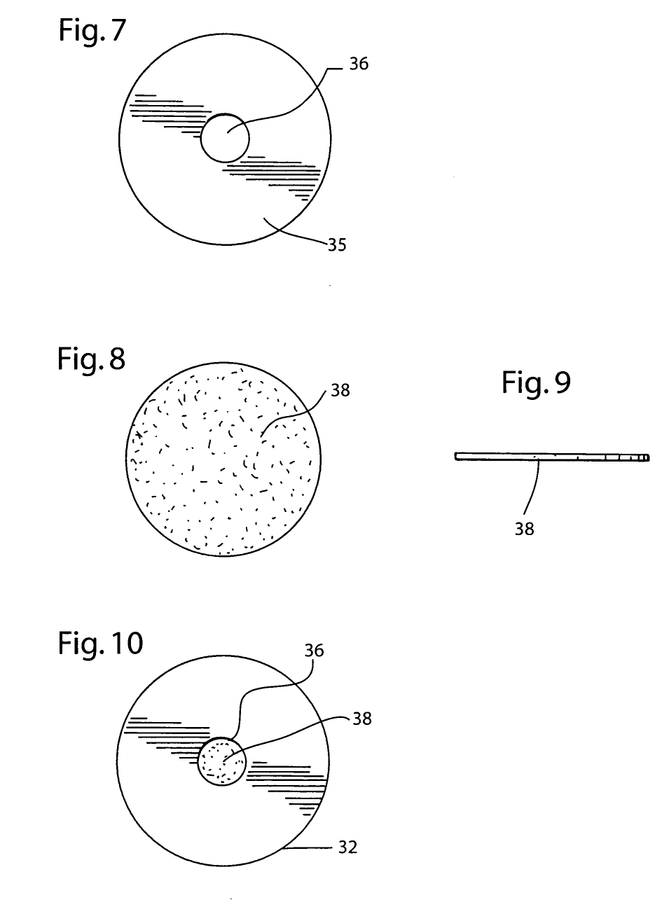

The preferred embodiment and best mode of the tissue form package invention is shown in FIGS. 1-10. The tissue package 10 comprises a blister pack 40 which holds a cylindrical container or jar 30, the blister pack outer flange 55 being covered with a cover 20. In the preferred embodiment of the present invention thin, plastic material is used that is sterilizable and can be formed by any suitable method such as for example, injection molding, pressure molding, vacuum forming and the like. The component material used for the blister pack of the allograft bone tissue package assembly 10 is preferably made in the form of a laminate made of an available material such as polytheylene terephthalateglycol (PETG) (a copolyester made by Eastman Chemical) as the inside layer, with the outside layer being another available material polycholrotrifluoroethylene (PCTFE) under the trademark ACLAR (a fluorinated-chlorinated thermoplastic made by Allied Corporation) which is impermeable to oxygen and other atmospheric gases and which is a highly moisture resistant barrier. The container cylindrical housing is preferably constructed of COC or any injection molded plastic that is compatible with freeze drying and the cap is constructed of polypropylene.

The outer container or blister pack 40 has a blister body 41 integrally formed with side walls 42, end walls 44 and a base 46 which together define an open faced cavity or chamber 48. The base 46 has a central section formed with a recess 47 which extends below the bottom of the base 46 to aid in the stability of the blister pack. A step 50 is formed in each end wall 44 with the lower section 45 of the end wall 44 being angled or sloped inward to the base 46. A protrusion 52 is formed in each end wall 44 on the lower portion 45 of the end wall 44 and extends inward into chamber 48 with the uppermost portion of protrusion 52 having a rounded end 53 which holds the jar in place. A flange portion 55 is disposed around the open chamber 48 and extends outward from the blister body 41. One end 56 of the flange 55 extends further out from the cavity 48. The end 56 is provided with a protrusion or embossed portion 58 which aids in the removal of the cover 20 from the planar surface of the flange 55.

The lid or cover 20 is sized and configured to cover the chamber 48 in its entirety and is seated on the upper planar surface of flange 55 continuously around the chamber 48 so that the peripheral edge 22 of the cover 20 is substantially aligned in an overlapping fashion with the peripheral edge 57 on the planar surfaced flange 55. The cover 20 is preferably an impermeable aluminum foil commercially available in 1 to 2 mil thickness sizes which bears product information.

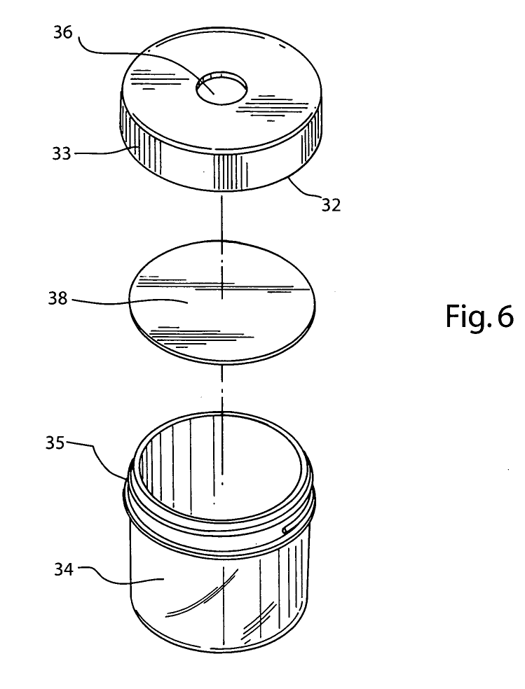

The inner cylindrical container 30 which is used for the allograft bone tissue material including chips, granules and powder as seen in FIG. 6 is molded from the COC plastic or other clear rigid medical plastic and is constructed of suitable dimensions so that it can be placed in the chamber 48 of the blister pack adjacent end spacer seating protrusion 52 with the cap 32 of the cylindrical container body 34 seated in the greater width section 64 and the body end seated in the lesser width section 62 to hold the container 30 firmly in place. The cap 32 is internally threaded to receive external threads 35 formed on the upper portion of cylindrical container body 34 allowing it to be removably mounted on the container body 34. The cap 32 is provided with a central vent hole 36 ranging from ⅜ inch diameter to 1 inch diameter with a preferred diameter of ½ inch and has serrations 33 on its outer rim to aid in twisting the cap, thus providing easy screwing or unscrewing of the same. A cylindrical breathable liner 38 with top and bottom planar surfaces is snap mounted inside the cap 32 so that its top planar surface is flush to the top inner surface of the cap covering vent hole 36. It is important that the material used for the liner 38 does not absorb lipids and discolor. The material found to meet the criteria noted above and to provide a microbial barrier is a sintered PTFE made by Porex, Inc. The insert liner 38 is preferably a disc 1 mm thick sintered PTFE with a pore size ranging from 1 to 10 microns with the preferred pore size ranging from 3-5 microns. Sintered PTFE is made by sintering together PTFE particles of a known size to create a strong material with a controlled pore size. Expanded PTFE is made with a stretching process that is weaker than sintered PTFE. The sintered PTFE provides a uniform porosity for consistent venting in a membrane that is stiff enough to be used ajar liner without needing a backing material. Expanded PTFE generally needs to be attached to a stiffer backing material such as polyethylene or polypropylene which can absorb lipids and discolor.

If desired the liner member 38 can be a two piece liner with a second liner section of TYVEK® or foamed PE with a 1 inch hole cut in the center to which a round disc of PTFE is bonded to the foamed PE disc to cover the hole. The end result is a one piece liner that provides adequate venting through the PTFE and an adequate seal between the foamed PE and the jar edge. PTFE and foamed PE do not absorb lipids while TYVEK® absorbs lipids and needs to combined with another liner material. The container 30 can be subjected to multiple freeze-thaw cycles through the very large temperature excursion required by the use of −80° C. freezing conditions. The temperature range change effecting the package could be as much as 110° C., from a −80° C. freezer to a +30° C. warehouse or storeroom area where the tissue may be selected for surgical use. Furthermore, the residual moisture of the allograft material was well below the maximum 6% residual moisture allowance generally between about 1.5% to about 2.2%.

A cover 20 as shown in FIGS. 1 and 2 is sealed to the surface of flange 55 once the container 30 with the allograft bone tissue material 100 has been mounted into the chamber 48. The material selected for the cover is foil which provides a sterile and moisture barrier and is easily sealed to the flange 55.

In the assembly of the invention, the implant tissue container 30 holding the implant material 100 is placed inside the chamber 48 so that the container 32 is held in place against the spacers 52 extending inward from the end walls 44 of the blister pack with the cap 32 positioned in the greater width section 64 defined by the side walls 42 with the bottom end of the container 32 mounted in the lesser width section 62 also defined by the sidewalls 42. The blister pack has a foil cover 20 heat sealed to the PETG outer planar surface of flange 55 sealing the chamber 48.

The packaging is easily disassembled during surgery by simply removing the cover and removing the container, unscrewing the container cap and depositing the implant bone tissue material for implantation into the patient. In addition using a clear packaging provides product visibility allowing easy identification of the product.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present inventions defined by the following claims.

What we claim is:

1. A container assembly for storing sterile allograft tissue implant material in a sterile condition comprising:
an outer container constructed with end walls, side walls and a base wall defining an open faced cavity and a flange extending outward from said cavity, said sidewalls defining a wider portion at one end and a narrower portion at the other end,
said end walls each defining a protruding member which extends into said cavity,
an implant container sized to fit into said inner container cavity, said implant container comprising a housing with a removable cap mounted thereto, said cap defining vent means therein and an insert liner member with a predetermined porosity sufficient to prevent microbial passage mounted in said cap covering said vent means.

2. A container assembly as claimed in claim 1 wherein outer container is a blister pack.

3. A container assembly as claimed in claim 1 wherein said flange has a protuberance formed thereon.

4. A container assembly as claimed in claim 1 wherein said end walls are angled inward and define a shelf.

5. A container assembly as claimed in claim 1 wherein said base wall defines a recess therein which extends outward from the rest of the base wall.

6. A container assembly as claimed in claim 1 wherein said cap vent means is a through going hole.

7. A container assembly as claimed in claim 1 wherein said insert liner member is constructed of sintered PTFE.

8. A container assembly as claimed in claim 7 wherein said insert liner member has a pore size ranging from 1-10 microns.

9. A container assembly as claimed in claim 8 wherein said insert liner member is about 1 mm thick with a pore size ranging from about 3 to about 5 microns.

10. A container assembly as claimed in claim 1 wherein said insert liner member is a two section construction, one section being PTFE and the other section being foamed PE.

11. A container assembly as claimed in claim 1 wherein said insert liner member is a two section construction, one section being PTFE and the other section being TYVEK®.

12. A container assembly as claimed in claim 1 wherein said side walls are angled inward and at least two sections have of different widths.

13. A container assembly as claimed in claim 1 wherein said end walls and said side walls are angled inward.

14. A container assembly as claimed in claim 1 wherein said flange of said outer container has one end which extends outward further than the other portions of said flange to form a handle for the respective container.

15. A container assembly as claimed in claim 14 wherein said handle has a rib formed thereon.

16. A package for storing sterile allograft bone tissue material comprising:
an outer container formed with end wall, side walls and a bottom member defining an open faced cavity, said side walls and said end walls being formed with a flange extending outward from said cavity,
said end walls each defining a protruding member which extends into said cavity and a step portion;
a cylindrical implant container sized to fit into said outer container cavity, said implant container comprising a cylindrical body with a removable cap mounted thereto, said cap defining a vent hole formed therein and a cylindrical porous insert member constructed of PTFE mounted in said cap covering said vent hole.

17. A container as claimed in claim 16 wherein said package container endwalls and side walls are angled inward so the upper surface of said cavity is greater than the area of said bottom.

18. A container as claimed in claim 16 wherein said insert member constructed of PTFE is sintered PTFE with a pore size ranging from 1-10 microns.

19. A container assembly as claimed in claim 16 wherein said bottom member defines a sink recess therein which extends past the outer wall of the rest of the bottom.

20. A container for sterile bone tissue material comprising:
a blister container housing defining an open faced cavity and a flange extending around said cavity outward from said cavity, said housing comprising a first end wall, side walls connected to said first end wall and second end wall, all of said walls being integrally connected with a base to form an interior cavity adapted to hold an implant container assembly and a flange extending from said walls;
an implant container assembly sized to fit into said blister container housing cavity, said implant container assembly comprising a cylindrical housing which can hold bone tissue implant material and a cap mounted on said housing, said cap defining at least one vent hole therein and an insert liner mounted in said cap adjacent said at least one vent hole; said insert liner being constructed of sintered PTFE with a pore size ranging from about 1-10 microns; and
a cover sealed to the flange of the container housing covering and sealing said container cavity.

21. A container as claimed in claim 20 wherein said blister container housing base defines a recess and said end walls define opposing projections extending into said cavity to engage and hold said implant container assembly.

22. A container as claimed in claim 20 wherein said bone tissue material is taken from a group consisting of powdered, granular and chip bone tissue.
